(19) United States
(12) Patent Application Publication
(10) Pub. No.: US 2022/0047303 A1
(43) Pub. Date: Feb. 17, 2022

(54) SYSTEMS AND METHODS FOR DELIVERING STIMULATION ELECTRODES TO ENDOCARDIAL OR OTHER TISSUE

(71) Applicant: EBR Systems, Inc., Sunnyvale, CA (US)

(72) Inventors: N. Parker Willis, Sunnyvale, CA (US); Timothy A. Fayram, Sunnyvale, CA (US); John Sam, Sunnvale, CA (US)

(21) Appl. No.: 17/404,502

(22) Filed: Aug. 17, 2021

Related U.S. Application Data
(60) Provisional application No. 63/066,699, filed on Aug. 17, 2020.

(51) Int. Cl. A61B 17/34; A61N 1/05; A61B 90/00

(52) U.S. Cl. CPC A61B 17/3468; A61N 1/056; A61B 2017/00044; A61B 2017/3486; A61B 2090/065; A61B 90/06

(57) ABSTRACT

The present technology is generally directed to delivery systems for medical implants, such as electrode assemblies for stimulating heart tissue. In some embodiments, a delivery system for a medical implant includes an elongate sheath having a distal portion and a balloon coupled to the distal portion of the sheath. The delivery system can further include a fluid circuit configured to be in fluid communication with the balloon and having a pressure source and a pressure sensor. The pressure source can move the balloon between an inflated configuration and a deflated configuration, and the pressure sensor can sense a pressure within the balloon. The sensed pressure can be monitored to determine (i) that the balloon is in contact with heart tissue of a heart, (ii), a motion profile of the heart tissue, and/or (iii) blood flow characteristics within the heart.

### CROSS-REFERENCE TO RELATED APPLICATION

This application claims the benefit of U.S. Provisional Patent Application No. 63/066,699, filed Aug. 17, 2020, and titled “SYSTEMS AND METHODS FOR ENDOCARDIAL CONTACT SENSING WITH A SHEATH BALLOON FOR WIRELESS ENDOCARDIAL PACING ELECTRODES,” which is incorporated herein by reference in its entirety.

### TECHNICAL FIELD

The present technology generally relates to methods and systems for stimulating cardiac tissue, and more particularly to systems and methods for delivering stimulation electrodes to endocardial or other tissues.

### BACKGROUND

Electrical stimulation of body tissue is used throughout medicine for treatment of both chronic and acute conditions. Among many examples, peripheral muscle stimulation is reported to accelerate healing of strains and tears, bone stimulation is likewise indicated to increase the rate of bone regrowth/repair in fractures, and nerve stimulation is used to alleviate chronic pain. Further there is encouraging research in the use of electrical stimulation to treat a variety of nerve and brain conditions, such as essential tremor, Parkinson's disease, migraine headaches, functional deficits due to stroke, and epileptic seizures.

Cardiac pacemakers and implantable defibrillators are examples of commonly implanted device utilizing electrical stimulation to stimulate cardiac and other tissues. A pacemaker is a battery-powered electronic device implanted under the skin, connected to the heart by an insulated metal lead wire with a tip electrode. Pacemakers were initially developed for and are most commonly used to treat slow heart rates (bradycardia), which may result from a number of conditions. More recently, advancements in pacemaker complexity, and associated sensing and pacing algorithms have allowed progress in using pacemakers for the treatment of other conditions, notably heart failure (HF) and fast heart rhythms (tachyarrhythmia/tachycardia).

Electrical energy sources connected to electrode/lead wire systems have typically been used to stimulate tissue within the body. The use of lead wires is associated with significant problems such as complications due to infection, lead failure, and electrode/lead dislodgement. The requirement for leads in order to accomplish stimulation also limits the number of accessible locations in the body. The requirement for leads has also limited the ability to stimulate at multiple sites (multisite stimulation).

Wireless stimulation electrodes are typically delivered via catheter-based delivery systems. One difficulty associated with implanting wireless stimulation electrodes is visualizing and determining the position of a distal end of a delivery catheter relative to a desired target location for implantation of the stimulation electrode. In particular, conventional delivery systems typically provide little or no tactile feedback that allows the implanter (e.g., a clinician) to know when the delivery catheter is close to or in contact with the target tissue. Accordingly, typical delivery methods include the use of fluoroscopic and/or echocardiographic techniques to provide visual feedback of the position and location of delivery catheter relative to the target tissue.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure.

FIG. 1 is a schematic diagram of a tissue stimulation system configured in accordance with embodiments of the present technology.

FIG. 2 is a partially schematic side view of a delivery system configured to facilitate delivery of an implantable medical device to tissue of a patient in accordance with embodiments of the present technology.

FIG. 3 is a side cross-sectional view of a distal portion of a sheath of the delivery system of FIG. 2 positioned within a heart chamber of a patient in accordance with embodiments of the present technology.

FIG. 4A is a schematic diagram of a sensed electrocardiogram (ECG) signal, a sensed electromyography (EMG) signal, and a sensed pressure of a balloon of the delivery system of FIG. 2 over time for a normal synchronous heart in accordance with embodiments of the present technology.

FIG. 4B is a side view of the distal portion of the sheath of the delivery system of FIG. 2 positioned within a heart chamber and illustrating synchronous contraction of the heart chamber for the normal synchronous heart in accordance with embodiments of the present technology.

FIG. 5A is a schematic diagram of a sensed ECG signal, a sensed EMG signal, and a sensed pressure of the balloon of the delivery system of FIG. 2 over time for an abnormal asynchronous heart in accordance with embodiments of the present technology.

FIGS. 5B and 5C are side views of the distal portion of the sheath of the delivery system of FIG. 2 positioned within a heart chamber and illustrating asynchronous contraction of the heart chamber for the abnormal asynchronous heart in accordance with embodiments of the present technology.

FIG. 6 is a flow diagram of a process or method for selecting a target site within a heart for electrical stimulation therapy in accordance with embodiments of the present technology.

### DETAILED DESCRIPTION

Aspects of the present disclosure are directed generally to systems and methods for delivering one or more stimulation electrodes to tissue of a patient, such as endocardial tissue, for implantation therein. In several of the embodiments described below, for example a delivery system for use in delivering a stimulation electrode can include (i) an elongate sheath having a distal portion, (ii) a balloon coupled to the distal portion of the sheath, and (iii) a fluid circuit configured to be in fluid communication with the balloon. The fluid circuit can include a pressure source (e.g., a syringe) that is fluidly connectable to the balloon and that is configured to inflate and deflate the balloon. The fluid circuit can further include a pressure sensor configured to monitor a pressure within the balloon.

The distal portion of the sheath and the balloon are configured to be advanced through the vasculature of a patient (e.g., a human patient) and into a heart chamber thereof, such as the left ventricle. The balloon can then be moved into contact with an endocardial wall of the heart chamber. When the balloon contacts the endocardial wall or is otherwise moved by the endocardial wall (e.g., during contraction thereof), the contact forces can displace the balloon-thereby causing pressure variations within the balloon that are detectable by the pressure sensor. In some embodiments, the delivery system can further include a computing device electrically coupled to the pressure sensor. The pressure sensor can convert the sensed pressure within the balloon to an electrical signal and pass the electrical signal to the computing device. The computing device can process the electrical signal to detect and analyze the pressure variations to determine, for example, (i) that the balloon is in contact with the wall of the heart chamber, (ii), a motion of the wall of the heart chamber, and/or (iii) blood flow characteristics within the heart chamber.

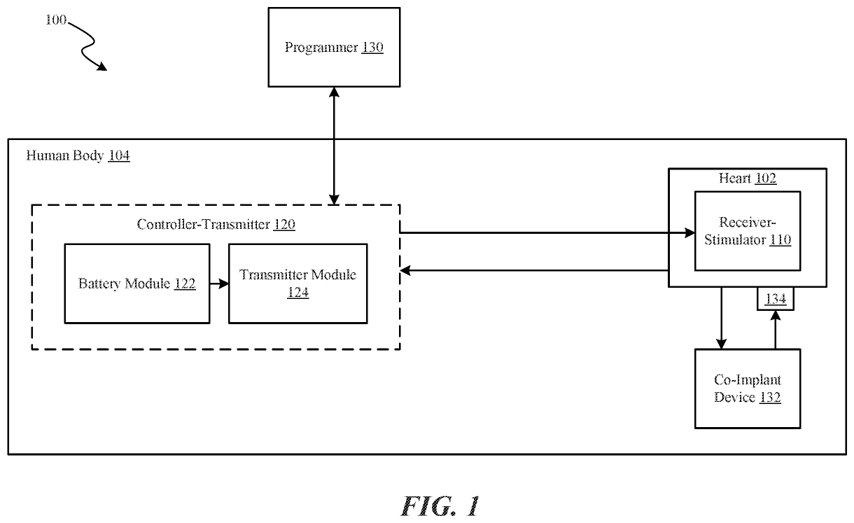

Specific details of several embodiments of the present technology are described herein with reference to FIGS. 1-6. The present technology, however, can be practiced without some of these specific details. In some instances, well-known structures and techniques often associated with leadless tissue stimulation systems, cardiac pacing, electronic circuitry, acoustic and radiofrequency transmission and receipt, delivery systems and catheters, and the like, have not been shown in detail so as not to obscure the present technology. Moreover, although many of the embodiments are described below with respect to systems and methods for left ventricular (LV) cardiac pacing, other applications and other embodiments in addition to those described herein are within the scope of the technology. For example, one of ordinary skill in the art will understand that one or more aspects of the present technology are applicable to other implantable devices configured to treat other areas of the human body.

The terminology used in the description presented below is intended to be interpreted in its broadest reasonable manner, even though it is being used in conjunction with a detailed description of certain specific embodiments of the disclosure. Certain terms can even be emphasized below; however, any terminology intended to be interpreted in any restricted manner will be overtly and specifically defined as such in this Detailed Description section.

The accompanying Figures depict embodiments of the present technology and are not intended to be limiting of its scope. The sizes of various depicted elements are not necessarily drawn to scale, and these various elements can be arbitrarily enlarged to improve legibility. Component details can be abstracted in the Figures to exclude details such as position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the spirit or scope of the present technology.

FIG. 1 is a schematic diagram of a tissue stimulation system 100 (“system 100”) configured in accordance with embodiments of the present technology. In the illustrated embodiment, the system 100 is configured to stimulate a heart 102 within a body 104 of a human patient. The system 100 can include one or more receiver-stimulators 110 (one shown in FIG. 1; which can also be referred to as stimulators, ultrasound receivers, stimulating electrodes, stimulation electrodes, acoustic receivers, and the like) in operable communication (e.g., wireless and/or radio communication) with a controller-transmitter 120 (which can also be referred to as an ultrasound transmitter, a pulse generator, an acoustic transmitter, and the like). The controller-transmitter 120 can include a battery module 122 and a transmitter module 124 operably coupled to and powered via the battery module 122. In some embodiments, both the receiver-stimulator 110 and the controller-transmitter 120 are configured to be implanted within the body 104 of the human patient. For example, the receiver-stimulator 110 can be implanted at and/or proximate the heart 102 (e.g., in the left ventricle, the right ventricle, or proximate area) for delivering stimulation pulses to the heart 102, while the controller-transmitter 120 can be positioned at another location remote from the heart 102 (e.g., in the chest area). In a particular embodiment, the receiver-stimulator 110 can be implanted within endocardial tissue of the left ventricle. The transmitter module 124 of the controller-transmitter 120 is configured to direct energy (e.g., acoustic energy, ultrasound energy) toward the receiver-stimulator 110, which is configured to receive the energy and deliver one or more electrical pulses (e.g., stimulation pulses, pacing pulses) to the heart 102.

In some embodiments, the system 100 can further include a programmer 130 in operable communication with the controller-transmitter 120. The programmer 130 can be positioned outside the body 104 and can be operable to program various parameters of the controller-transmitter 120 and/or to receive diagnostic information from the controller-transmitter 120. In some embodiments, the system 100 can further include a co-implant device 132 (e.g., an implantable cardioverter defibrillator (ICD) or pacemaker) coupled to pacing leads 134 for delivering stimulation pulses to one or more portions of the heart 102 other than the area stimulated by the receiver-stimulator 110. In other embodiments, the co-implant device 132 can be a leadless pacemaker which is implanted directly into the heart 102 to eliminate the need for separate pacing leads 134. The co-implant device 132 and the controller-transmitter 120 can be configured to operate in tandem and deliver stimulation signals to the heart 102 to cause a synchronized heartbeat. In some embodiments, the controller-transmitter 120 can receive signals (e.g., electrocardiogram signals) from the heart 102 to determine information related to the heart 102, such as a heart rate, heart rhythm, including the output of the pacing leads 134 located in the heart 102. In some embodiments, the controller-transmitter 120 can alternatively or additionally be configured to receive information (e.g., diagnostic signals) from the receiver-stimulator 110. The received signals can be used to adjust the ultrasound energy signals delivered to the receiver-stimulator 110.

The receiver-stimulator 110, the controller-transmitter 120, and/or the programmer 130 can include a machine-readable (e.g., computer-readable) or controller-readable medium containing instructions for generating, transmitting, and/or receiving suitable signals (e.g., stimulation signals, diagnostic signals). The receiver-stimulator 110, the controller-transmitter 120, and/or the programmer 130 can include one or more processor(s), memory unit(s), and/or input/output device(s). Accordingly, the process of providing stimulation signals and/or executing other associated functions can be performed by computer-executable instructions contained by, on, or in computer-readable media located at the receiver-stimulator 110, the controller-transmitter 120, and/or the programmer 130. Further, the receiver-stimulator 110, the controller-transmitter 120, and/or the programmer 130 can include dedicated hardware, firmware, and/or software for executing computer-executable instructions that, when executed, perform any one or more methods, processes, and/or sub-processes described herein. The dedicated hardware, firmware, and/or software also serve as “means for” performing the methods, processes, and/or sub-processes described herein.

In some embodiments, the system 100 can include several features generally similar or identical to those of the leadless tissue stimulation systems disclosed in (i) U.S. Pat. No. 7,610,092, filed Dec. 21, 2005, and titled “LEADLESS TISSUE STIMULATION SYSTEMS AND METHODS,” (ii) U.S. Pat. No. 8,315,701, filed Sep. 4, 2009, and titled “LEADLESS TISSUE STIMULATION SYSTEMS AND METHODS,” and/or (iii) U.S. Pat. No. 8,718,773, filed May 23, 2007, and titled “OPTIMIZING ENERGY TRANSMISSION IN A LEADLESS TISSUE STIMULATION SYSTEM.”

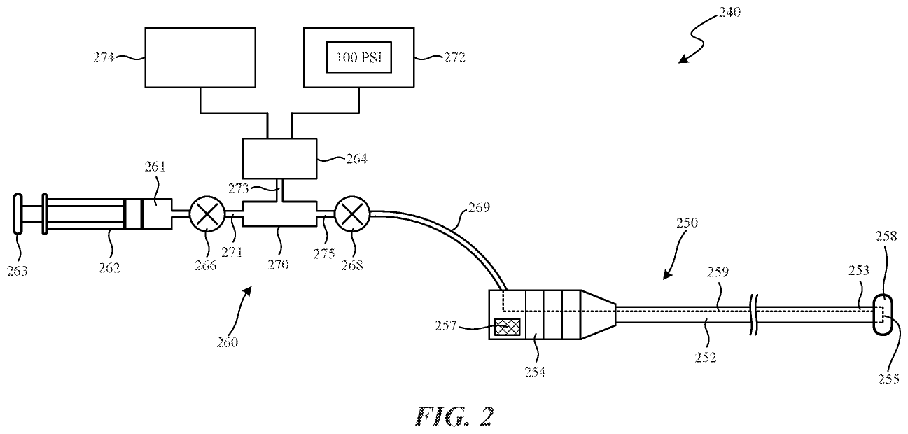

FIG. 2 is a partially schematic side view of a delivery system 240 configured to facilitate delivery of an implantable medical device to tissue of a patient in accordance with embodiments of the present technology. In some embodiments, the medical device can include one or more of the receiver-stimulators 110 of FIG. 1 and the tissue can be cardiac tissue of the patient, such as endocardial tissue of the left ventricle. In the illustrated embodiment, the delivery system 240 includes a sheath assembly 250 including an elongate sheath 252 (which can also be referred to as a shaft, catheter, elongate member, and the like) coupled to a handle 254. The sheath 252 includes a distal portion 253 including a distal tip or terminus 255. The handle 254 can include features for manipulating/steering the sheath 252, such as through the vasculature of a patient and into the heart (e.g., the left ventricle) of the patient. In some embodiments, the handle 254 can include one or more lumens, ports, valves and/or the like for facilitating insertion of a medical instrument (e.g., a delivery catheter) through the handle 254 and into a lumen of the sheath 252.

In the illustrated, the sheath assembly 250 further includes a balloon 258 coupled to the distal portion 253 of the sheath 252. In some embodiments, the balloon 258 can be secured around an entire periphery (e.g., circumference) of the distal portion 253 of the sheath 252. The balloon 258 can be formed of a flexible, compliant material and can be inflated from a deflated (e.g., collapsed) configuration to an inflated (e.g., expanded) configuration shown in FIG. 2, as described in greater detail below. In some embodiments, when inflated, the balloon 258 can project distally past the distal tip 255 of the sheath 252. In some embodiments, the handle 254 and/or another component of the delivery system 240 can include an indicator 257 (e.g., a display, a light) configured to indicate that the balloon 258 is in the inflated configuration at a selected (e.g., pre-selected) inflation pressure.

In the illustrated embodiment, the balloon 258 is operably coupled to an inflation and monitoring circuit 260 via a fluid line 259 extending through the sheath 252 and the handle 254. The fluid line 259 can include one or more tubes, pipes, lumens, and/or the like fluidly connecting the balloon 258 to the inflation and monitoring circuit 260. The inflation and monitoring circuit 260 can include a pressure source 262 and a pressure sensor 264 (e.g., a pressure transducer) configured to be in fluid communication within the balloon 258 via the fluid line 259. More specifically, in the illustrated embodiment the inflation and monitoring circuit 260 includes a connector 270 (e.g., a T-connector) having (i) a first branch 271 configured to be in fluid communication with the pressure source 262 via a first fluid control device 266, (ii) a second branch 273 fluidly connected to the pressure sensor 264, and (iii) a third branch 275 configured to be in fluid communication with the fluid line 259 via a second fluid control device 268 and a conduit 269 extending between the second fluid control device 268 and the fluid line 259. In other embodiments, the pressure source 262 and the pressure sensor 264 can be fluidly connected to the balloon 258 in other manners, such as via different combinations of fluid control devices, connectors, conduits, and the like.

In the illustrated embodiment, the pressure source 262 is a syringe having a plunger 263. In other embodiments, the pressure source 262 can be a pump, blower, and/or other suitable pressure source. The pressure source 262 (e.g., the plunger 263) is configured to drive a fluid 261 through the inflation and monitoring circuit 260 toward/away from the balloon 258 to inflate/deflate the balloon 258. The fluid 261 can be a saline solution, air, and/or other gas or liquid. In some embodiments, the fluid 261 can include a contrast agent visible via an imaging system (e.g., a fluoroscopic imaging system). For example, the fluid 261 can be a 50%-50% mixture of normal saline (e.g., distilled H₂O and 0.9% NaCl) and an X-ray dye contrast agent. In some embodiments, the first and second fluid control devices 266, 268 are stopcocks or other components manually actuatable by a user or automatically actuatable to fluidly connect the pressure source 262 to the connector 270 and the connector 270 to the conduit 269, respectively. The pressure sensor 264 can be a digital or manual device for quantifying an inflation pressure of the balloon 258. In some embodiments, the pressure sensor 264 can be one or more of the types manufactured by Opsens Solutions of Quebec City, Canada. In some embodiments, the pressure sensor 264 can be sensitive enough to detect pressure changes within the balloon 258 at the millibar level.

In some embodiments, the pressure sensor 264 can be operably coupled (e.g., via an electrical connection, wireless connection, wired connection) to a digital pressure readout 272 and/or a computing device 274 (e.g., a processing device). The digital readout 272 can be configured to display a readout of the inflation pressure of the balloon 258 (e.g., “100 PSI”) determined by the pressure sensor 264. The computing device 274 can be a computer, laptop, tablet, smartphone, etc., and can comprise a processor and a non-transitory computer-readable storage medium that stores instructions that when executed by the processor, carry out the functions attributed to the computing device as described herein. Although not required, aspects and embodiments of the present technology can be described in the general context of computer-executable instructions, such as routines executed by a general-purpose computer, e.g., a server or personal computer. Those skilled in the relevant art will appreciate that the present technology can be practiced with other computer system configurations, including Internet appliances, hand-held devices, wearable computers, cellular or mobile phones, multi-processor systems, microprocessor-based or programmable consumer electronics, set-top boxes, network PCs, mini-computers, mainframe computers and the like. The present technology can be embodied in a special purpose computer or data processor that is specifically programmed, configured or constructed to perform one or more of the computer-executable instructions explained in detail below. Indeed, the term “computer” (and like terms), as used generally herein, refers to any of the above devices, as well as any data processor or any device capable of communicating with a network, including consumer electronic goods such as game devices, cameras, or other electronic devices having a processor and other components, e.g., network communication circuitry.

The present technology can also be practiced in distributed computing environments, where tasks or modules are performed by remote processing devices, which are linked through a communications network, such as a Local Area Network (“LAN”), Wide Area Network (“WAN”), or the Internet. In a distributed computing environment, program modules or sub-routines can be located in both local and remote memory storage devices. Aspects of the present technology described below can be stored or distributed on computer-readable media, including magnetic and optically readable and removable computer discs, stored as in chips (e.g., EEPROM or flash memory chips). Alternatively, aspects of the present technology can be distributed electronically over the Internet or over other networks (including wireless networks). Those skilled in the relevant art will recognize that portions of the present technology can reside on a server computer, while corresponding portions reside on a client computer. Data structures and transmission of data particular to aspects of the present technology are also encompassed within the scope of the present technology.

In operation, a user (e.g., a physician or other clinician) or an automated device (e.g., the computing device 274) can inflate the balloon 258 by opening the first and second fluid control devices 266, 268 and depressing the plunger 263 of the syringe 262 to increase the pressure in the balloon 258 (and the intermediate components of the delivery system 240 such as the connector 270, the conduit 269, and the fluid line 259) by forcing a portion of the fluid 261 from the syringe 262 into the inflation and monitoring circuit 260. Similarly, the user can deflate the balloon 258 by opening the first and second fluid control devices 266, 268 and withdrawing the plunger 263 of the syringe 262 to decrease the pressure in the balloon 258 by drawing a portion of the fluid 261 from the inflation and monitoring circuit 260 into the syringe 262. Closing the first fluid control device 266 can fluidly disconnect the pressure source 262 from the balloon 258 and the inflation and monitoring circuit 260, thereby fixing (e.g., locking, setting) the inflation pressure of the balloon 258 at a constant pressure (e.g., “100 PSI”). With the first fluid control device 266 closed and the second fluid control device 268 open, the pressure sensor 264 can sense/monitor the constant inflation pressure of the balloon 258 and can also detect any pressure changes attributable to external forces acting on the balloon 258.

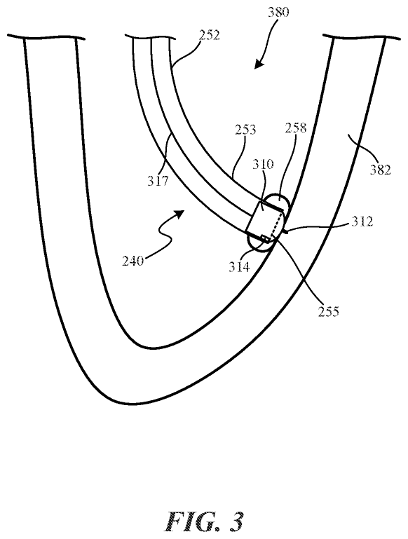

More specifically, for example, FIG. 3 is a side cross-sectional view of the distal portion 253 of the sheath 252 of the delivery system 240 of FIG. 2 positioned within a heart chamber 380 of a patient in accordance with embodiments of the present technology. In some embodiments, the heart chamber 380 can be the left ventricle of a human patient and can include an endocardial wall 382 (e.g., a left ventricular wall).

In the illustrated embodiment, the delivery system 240 further includes a receiver-stimulator 310 (omitted in FIG. 2) positioned at the distal portion 253 of the sheath 252. In some embodiments, the receiver-stimulator 310 can be attached to the distal tip 255 of the sheath 252 and/or can be advanced through the lumen of the sheath 252 from the handle 254 (FIG. 2). The receiver-stimulator 310 can include a cathode 312 (e.g., a stimulation electrode) and an anode 314 for stimulating tissue of the endocardial wall 382. The cathode 312 can be located at the distal tip of the receiver-stimulator 310 and can have a smaller surface area than the anode 314. In some embodiments, the cathode 312 can project distally past the distal tip 255 of the sheath 252 and/or the balloon 258. During delivery, the receiver-stimulator 310 can be temporarily electrically coupled to an external monitor and pacing controller via one or more conductive lines 317 routed through the sheath 252 to allow for externally controlled monitoring and pacing. In some embodiments, the delivery system 240 and the receiver-stimulator 310 can include some features that are at least generally similar in structure and function, or identical in structure and function, to those of the delivery systems and receiver-stimulators disclosed in U.S. Pat. No. 9,283,392, filed Sep. 24, 2010, and titled “TEMPORARY ELECTRODE CONNECTION FOR WIRELESS PACING SYSTEMS,” which is incorporated herein by reference in its entirety.

In the illustrated embodiment, the balloon 258 is inflated and the delivery system 240 is positioned such that (i) the balloon 258 is positioned against the endocardial wall 382 and (ii) the receiver-stimulator 310 contacts (e.g., electrically contacts, is inserted in) the endocardial wall 382. In some embodiments, the sheath 252 can be advanced into the heart chamber 380 with the balloon 258 in the deflated configuration, and the balloon 258 can then be inflated within the heart chamber 380 before being advanced toward the endocardial wall 382. Referring to FIGS. 2 and 3 together, in such embodiments the pressure sensor 264 can sense when the delivery system 240 contacts the endocardial wall 382. Specifically, the pressure of the balloon 258 detected by the pressure sensor 264 can spike when the balloon 258 initially contacts the endocardial wall 382, which temporarily displaces the flexible balloon 258 and reduces the volume of the balloon 258. The displacement causes one or more back pressure waves (e.g., pressure variations) to travel through the fluid 261 from the balloon 258, through the fluid line 259, and to the inflation and monitoring circuit 260 and the pressure sensor 264. In some embodiments, the computing device 274 can process the electrical signal(s) from the pressure sensor 264 to detect that the delivery system 240 has contacted the endocardial wall 382 based on the pressure variations. For example, the computing device 274 can detect variations in the amplitude of the signals from the pressure sensor 264 and/or variations in frequency (harmonic or non-harmonic) of the signals that indicate endocardial wall contact (e.g., using a fast Fourier transform algorithm).

In some embodiments, the computing device 274 can further process the signals from the pressure sensor 264 to determine (e.g., quantify) a contact force at which the balloon 258 contacts the endocardial wall 382. The contact force can be used to determine whether the delivery system 240 is in proper contact or improper contact with the endocardial wall 382 by, for example, comparing the determined contact force to a preselected or predetermined desired contact force. For example, an improper contact force can be determined as (i) a force above a preselected force at which implantation of the receiver-stimulator 310 might result in the receiver-stimulator 310 being positioned too deep within the tissue of the endocardial wall 382 and/or (ii) a force below a preselected force at which implantation of the receiver-stimulator 310 might result in the receiver-stimulator 310 being positioned too shallow within the tissue of the endocardial wall 382.

In some embodiments, the delivery system 240 can provide one or more indications to the user (e.g., a physician implanting the receiver-stimulator 310) that the delivery system 240 has properly contacted the endocardial wall 382. For example, the delivery system 240 can be configured to provide tactile feedback, visual feedback, and/or auditory feedback to the user that contact has occurred and/or that the contact is at or proximate the desired contact force. In some embodiments, the indicator 257 and/or one or more additional display devices (e.g., indicator lights; not shown) can illuminate in different colors (e.g., red, blue, yellow) and/or in different patterns (e.g., constant light, blinking light) to indicate proper contact, no contact, and/or improper contact.

Accordingly, in some aspects of the present technology the delivery system 240 is configured to provide an indication of endocardial wall contact to the user. This indication can be helpful to the user because the sheath 252 and the receiver-stimulator 310 may provide little or no tactile feedback that allows the user to know when they are close to or in contact with the endocardial wall 382. Therefore, by detecting and quantifying endocardial wall contact, the delivery system 240 can decrease the likelihood of implanting the receiver-stimulator 310 at (i) too shallow of a depth at which stimulation may not be effective or (ii) too deep a depth at which the receiver-stimulator 310 could puncture the endocardial wall 382—thereby increasing the likelihood of successfully implanting the receiver-stimulator 310 at a target position and depth where stimulation therapy is likely to be effective. In contrast to the present technology, some cardiac pacing technologies use fluoroscopy and/or echocardiography (e.g., intracardiac or transesophageal echocardiography) to provide visual feedback of the position and location of a sheath and receiver electrode catheter system relative to an endocardial wall and a target implant position. However, such techniques require expensive and complicated additional machinery and imaging techniques, and the visual feedback from such systems can become occluded.

In addition to sensing endocardial wall contact, in some embodiments the computing device 274 can process the signals from the pressure sensor 264 to determine one or more characteristics of the heart chamber 380. For example, movement of the endocardial wall 283 and/or blood flow (e.g., pulsatile blood flow during depolarization) through the heart chamber 380 can act against the balloon 258 to create detectable pressure changes within the balloon 258. For example, as the endocardial wall 382 contracts and relaxes, the pressure in the balloon 258 can vary creating a pulsatile waveform. Accordingly, such pressure variations can be processed to determine mechanical motion characteristics of and/or blood flow characteristics within the heart chamber 380.

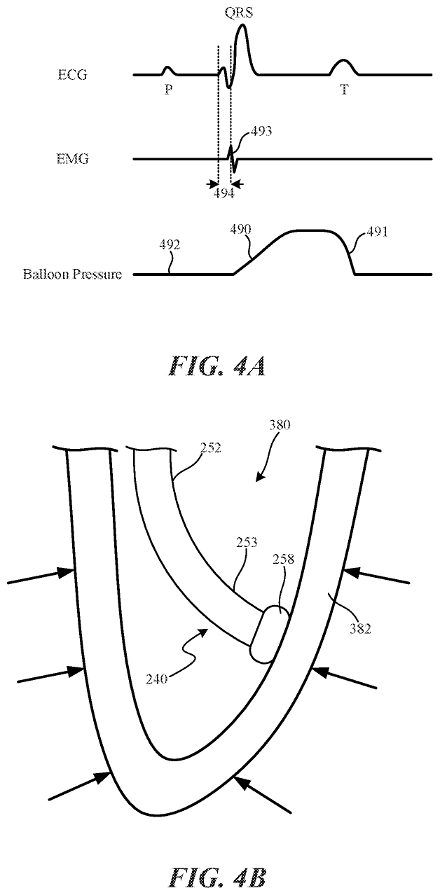

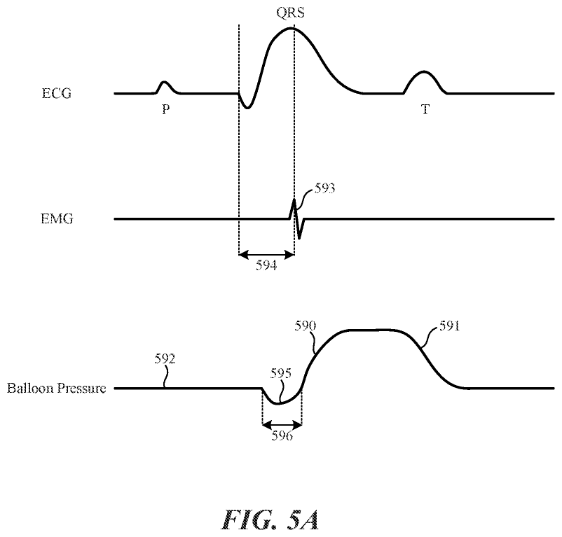

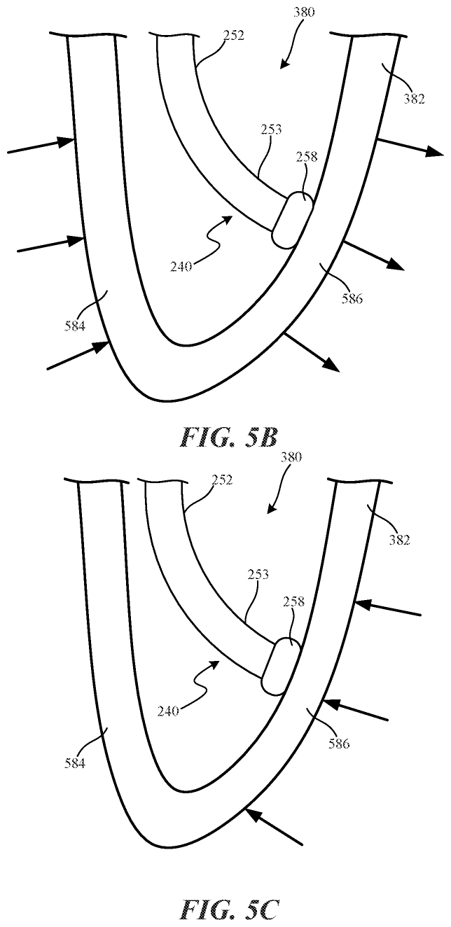

In some embodiments, pressure changes in the balloon 258 from mechanical motion of the heart chamber 380 can be used—in addition to or instead of electrical information detected from the heart—to help detect abnormal motion of the endocardial wall 382 and/or to help select a target site for implantation of the receiver-stimulator 310 along the endocardial wall 382. More specifically, for example, FIG. 4A is a schematic diagram of a sensed electrocardiogram (ECG) signal, a sensed electromyography (EMG) signal, and a sensed pressure of the balloon 258 over time for a normal (e.g., healthy) synchronous heart in accordance with embodiments of the present technology. FIG. 4B is a side view of the distal portion 253 of the sheath 252 of the delivery system 240 of FIG. 2 positioned within the heart chamber 380 and illustrating synchronous contraction of the heart chamber 380 for the normal synchronous heart in accordance with embodiments of the present technology. FIG. 5A is a schematic diagram of a sensed ECG signal, a sensed EMG signal, and a sensed pressure of the balloon 258 over time for an abnormal (e.g., diseased) asynchronous heart in accordance with embodiments of the present technology. FIGS. 5B and 5C are side views of the distal portion 253 of the sheath 252 of the delivery system 240 of FIG. 2 positioned within the heart chamber 380 and illustrating two stages of asynchronous contraction of the heart chamber 380 for the abnormal asynchronous heart in accordance with embodiments of the present technology. In some embodiments, the receiver-stimulator 310 (obscured in FIGS. 4A, 5B, and 5C; shown in FIG. 3) can be configured to sense the ECG signals and/or the EMG signals and to pass the ECG signals and/or the EMG signals to the external monitor and pacing controller and/or the computing device 274 (FIG. 2) via the conductive lines 317 (FIG. 3). In some embodiments, the controller-transmitter 120 (FIG. 1), the external monitor and pacing controller, and/or another source external can directly detect the ECG signals (e.g., surface ECG signals).

Referring first to FIGS. 4A and 4B together, in the normal synchronous heart, the heart chamber 380 synchronously contracts (as indicated by the inward arrows in FIG. 4B) creating a single monophasic pulse. The contraction is represented in the ECG signal as the QRS complex which measures depolarization of the heart chamber 380. As the heart chamber 380 contracts, the endocardial wall 382 acts against (e.g., depresses) the inflated balloon 258 to increase the pressure within the balloon 258 as shown by a segment 490 of the plot of “Balloon Pressure.” Accordingly, there is no or little delay between detection of (i) the electrical signals of the heart (e.g., the QRS complex of the ECG signal) (ii) and mechanical contraction of the heart chamber 380 (e.g., as indicated by the increased pressure in the balloon 258 at the segment 490). As the heart chamber 380 subsequently repolarizes and relaxes, the pressure within the balloon 258 decreases (e.g., returns; as shown by a segment 491 of the plot of “Balloon Pressure”) to a baseline, selected inflation pressure 492 (e.g., about 100 PSI).

The EMG signal can provide an electrical analog to the balloon pressure signal. For example, as shown in FIG. 4A, the EMG signal includes a spike or waveform 493 indicating that the portion of the endocardial wall 382 adjacent the delivery system 240 (e.g., adjacent the receiver-stimulator 310 of FIG. 3) has been electrically activated. For the normal heart represented in FIG. 4A, an interval 494 (e.g., a left ventricular delay (QLV) interval) between the onset of the QRS complex of the ECG signal and the waveform 493 of the EMG signal (e.g., a first large positive or negative peak of the EMG signal) can be relatively small. Accordingly, the EMG signal and the balloon pressure signal can each provide an indication of local electrical activation and corresponding mechanical motion of the heart chamber 380 relative to the surface (e.g., global) electrical signals of the heart chamber 380 detected in the ECG signal.

Referring next to FIGS. 5A-5C together, in the abnormal asynchronous heart, a first portion 584 of the endocardial wall 382 can contract (as indicated by the inward arrows in FIG. 5B) before a second portion 586 of the endocardial wall 382 adjacent the delivery system 340 contracts (as indicated by the inward arrows in FIG. 5C). Contraction of the first portion 584 of the endocardial wall 382 before the second portion 586 can cause the second portion 586 and/or other portions of the heart chamber 380 to bulge outward as indicated by the outward arrows in FIG. 5B. In some embodiments, such asynchronous biphasic contraction of the left ventricle can be a symptom of heart failure. In such embodiments, as shown by a segment 595 of the plot of “Balloon Pressure,” the detected pressure of the balloon 258 can initially decrease from a baseline inflation pressure 592 as the second portion 586 bulges outward (FIG. 5B) and reduces its contact force against the balloon 258. Then, as the second portion 586 contracts, the second portion 586 can depress the inflated balloon 258 to increase the pressure within the balloon 258 as shown by a segment 590 of the plot of “Balloon Pressure.” Accordingly, there balloon pressure signal can capture a mechanical delay 596 between the contraction of the first and second portions 584, 586 of the endocardial wall 382. Finally, the pressure within the balloon 258 decreases (e.g., returns; as shown by a segment 591 of the plot of “Balloon Pressure”) to the selected inflation pressure 592 as the heart chamber 380 repolarizes and relaxes.

As described above, the EMG signal can provide an electrical analog to the balloon pressure signal. For example, as shown in FIG. 5A, the EMG signal includes a spike or waveform 593 indicating that the second portion 586 of the endocardial wall 382 adjacent the delivery system 240 (e.g., adjacent the receiver-stimulator 310 of FIG. 3) has been electrically activated. For the abnormal heart represented in FIG. 5A, an interval 594 (e.g., a QLV interval) between the onset of the QRS complex of the ECG signal and the waveform 593 of the EMG signal can be relatively large-indicating the abnormal biphasic contraction of the heart chamber 380.

Accordingly, in some aspects of the present technology the pressure of the balloon 258 can provide an indication that a region of the endocardial wall 382 adjacent the delivery system 240 (e.g., the second portion 386) moves abnormally during the cardiac cycle as indicated, for example, by the delay 596. In some embodiments, such regions are good candidates for implantation of the receiver-stimulator 310 (FIG. 3) and subsequent cardiac stimulation therapy (e.g., left-side pace making for cardiac resynchronization therapy). In particular, it is expected that implantation sites with longer mechanical delays (e.g., as indicated by the delay 596) will provide for more effective stimulation treatment.

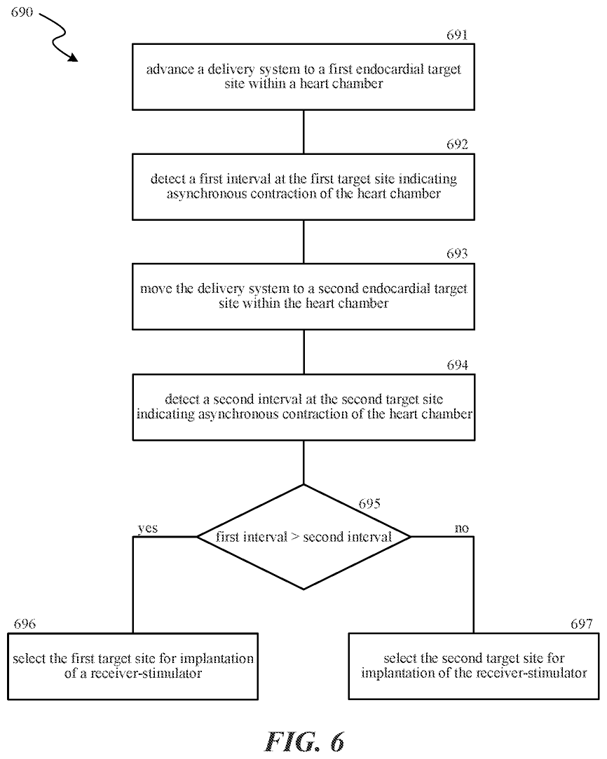

FIG. 6 is a flow diagram of a process or method 690 for selecting a target site within a heart for electrical stimulation therapy in accordance with embodiments of the present technology. Although some features of method 690 are described in the context of the embodiments described in detail with reference to FIGS. 1-5C, one skilled in the art will readily understand that the method 690 can be carried out using other suitable systems and/or devices described herein.

At block 691, the method 690 includes advancing the delivery system 240 to a first target site along the endocardial wall 382 of the heart chamber 380. For example, the balloon 258 can be inflated and advanced into contact with the endocardial wall 382 at the first target site. At block 692, the method 690 includes detecting a first interval at the first target site indicating asynchronous contraction of the heart chamber 380. The first interval can be the interval 594 determined from the EMG signal and/or the delay 596 determined from the balloon pressure signal. At block 693, the method 690 includes moving the delivery system 240 to a second target site along the endocardial wall 382. At block 694, the method 690 includes detecting a second interval at the second target site indicating asynchronous contraction of the heart chamber 380. The second interval can be the interval 594 determined from the EMG signal and/or the delay 596 determined from the balloon pressure signal.

At block 695, the method 690 includes comparing the first interval to the second interval. If the first interval is greater than the second interval, the method 690 can include selecting the first target site for implantation of the receiver-stimulator 310. If the second interval is greater than the first interval, the method 690 can include selecting the second target site for implantation of the receiver-stimulator 310. In some embodiments, the selection can be based on both the intervals 594 determined from the EMG signal and the delays 596 determined from the balloon pressure signal at blocks 692 and 694 such that the target site is selected based on both electrical information of the heart (from the EMG signal) and mechanical motion information of the heart (from the balloon pressure signal). Moreover, in some embodiments more than two different target sites can be compared to determine a target site with the longest interval.

In contrast to the present technology, determining sections of a heart chamber with abnormal wall motion typically requires performing an echocardiogram strain study on a patient. Such studies require pre-testing the patient by performing the echocardiogram strain study before a surgical implant procedure, and then ensuring during the surgical procedure that the location of the implant matches the target location identified in the pre-testing echocardiogram strain study. Accordingly, in some aspects of the present technology the delivery system 240 can be used to intraoperatively determine abnormal wall motion at multiple target sites-thereby reducing or eliminating the need for preoperative tests, such as echocardiograms, and making it easier to ensure that an implant is at a correct target site within the heart chamber.

The following examples are illustrative of several embodiments of the present technology:

1. A delivery system for a medical implant, comprising:
an elongate sheath having a distal portion;
a balloon coupled to the distal portion of the sheath; and
a fluid circuit configured to be in fluid communication with the balloon, wherein the fluid circuit includes—
   a pressure source configured to move the balloon between an inflated configuration and a deflated configuration; and
   a pressure sensor configured to sense a pressure within the balloon.

2. The delivery system of example 1 wherein the distal portion of the sheath includes a distal terminus, and wherein the balloon extends distally past the distal terminus in the inflated configuration.

3. The delivery system of example 1 or example 2 wherein—
the fluid circuit further includes a connector, a first fluid control device, and a second fluid control device,
the first fluid control device is between the pressure source and the connector and actuatable to fluidly connect the pressure source to the connector,
the pressure sensor is fluidly coupled to the connector, and
the second fluid control device is between the connector and the balloon and actuatable to fluidly connect the connector to the balloon.

4. The delivery system of any one of examples 1-3 wherein the pressure source is a syringe.

5. The delivery system of any one of examples 1-4 wherein the elongate sheath is sized and shaped to be advanced into a heart chamber of a patient such that the balloon contacts a wall of the heart chamber.

6. The delivery system of example 5, further comprising a computing device electrically coupled to the pressure sensor, wherein—
the pressure sensor is configured to convert the sensed pressure within the balloon to an electrical signal,
the computing device is configured to receive the electrical signal from the pressure sensor, and
the computing device is configured to process the electrical signal to determine that the balloon is in contact with the wall of the heart chamber.

7. The delivery system of example 5 or example 6, further comprising a computing device electrically coupled to the pressure sensor, wherein—
the pressure sensor is configured to convert the sensed pressure within the balloon to an electrical signal,
the computing device is configured to receive the electrical signal from the pressure sensor, and
the computing device is configured to process the electrical signal to determine a motion of the wall of the heart chamber.

8. The delivery system of any one of examples 5-7, further comprising a computing device electrically coupled to the pressure sensor, wherein—
the pressure sensor is configured to convert the sensed pressure within the balloon to an electrical signal,
the computing device is configured to receive the electrical signal from the pressure sensor, and
the computing device is configured to process the electrical signal to determine a blood flow characteristic within the heart chamber.

9. The delivery system of any one of examples 1-8, further comprising an indicator operably coupled to the pressure sensor, wherein the indicator is configured to provide an audible and/or visual indication that the pressure within the balloon is at a selected inflation pressure.

10. A method of sensing contact of a delivery system with a wall of a heart chamber, the method comprising:
advancing a distal portion of a sheath of the delivery system into the heart chamber;
inflating a balloon coupled to the distal portion of the sheath;
advancing the distal portion of the sheath and the balloon toward the wall;
monitoring a pressure within the balloon; and
determining that the delivery system has contacted the wall by sensing a change in pressure within the balloon.

11. The method of example 10 wherein sensing the change in pressure within the balloon includes sensing an increase in pressure within the balloon.

12. The method of example 10 or example 11 wherein monitoring the pressure within the balloon includes fluidly coupling a digital pressure sensor to the balloon.

13. The method of any one of examples 10-2 wherein inflating the balloon includes fluidly connecting a syringe to the balloon and actuating the syringe to drive a fluid into the balloon, and wherein monitoring the pressure within the balloon further includes fluidly disconnecting the syringe from the balloon after inflating the balloon.

14. The method of any one of examples 10-13 wherein inflating the balloon includes inflating the balloon to a selected inflation pressure of about 100 pounds per square inch.

15. The method of any one of examples 10-14 wherein the method further comprises providing a visual and/or auditory indication that the delivery system has contacted the wall.

16. A method of selecting a target site of an endocardial wall for a medical implant, the method comprising:
inflating a balloon coupled to a distal portion of a sheath;
moving the balloon into contact with the endocardial wall at a first site;
monitoring a pressure within the balloon to detect a first delay associated with contraction of the endocardial wall at the first site;
moving the balloon into contact with the endocardial wall at a second site;
monitoring the pressure within the balloon to detect a second delay associated with contraction of the endocardial wall at the second site; and
comparing the first delay and the second delay to select the first site or the second site as the target site.

17. The method of example 16 wherein comparing the first delay to the second delay to select the first site or the second site as the target site includes determining the greater of the first delay and the second delay and selecting the first site or the second site as the target site based on the greater of the first delay and the second delay.

18. The method of example 16 or example 17 wherein monitoring the pressure within the balloon to detect the first delay includes sensing a first decrease in the pressure within the balloon, and wherein monitoring the pressure within the balloon to detect the second delay includes sensing a second decrease in the pressure within the balloon.

19. The method of any one of examples 16-18 wherein the method further comprises:
sensing a surface electrocardiogram (ECG) signal;
sensing an electromyography (EMG) signal;
when the balloon is in contact with the endocardial wall at the first site, determining a first interval between a first QRS complex of the ECG signal and a first waveform of the EMG signal;
when the balloon is in contact with the endocardial wall at the first site, determining a second interval between a second QRS complex of the ECG signal and a second waveform of the EMG signal; and comparing the first interval and the second interval to further select the first site or the second site as the target site.

20. The method of any one of examples 16-19 wherein the endocardial wall is within the left ventricle.

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments can perform steps in a different order. Likewise, the various electronic components and functions can be separated into more or fewer electronic circuit elements and/or functional blocks. The various components and/or functionalities of the embodiments described herein can also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms can also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications can be made without deviating from the technology. Further, while advantages associated with some embodiments of the technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein.

I/We claim:

1. A delivery system for a medical implant, comprising:
an elongate sheath having a distal portion;
a balloon coupled to the distal portion of the sheath; and
a fluid circuit configured to be in fluid communication with the balloon, wherein the fluid circuit includes—
a pressure source configured to move the balloon between an inflated configuration and a deflated configuration; and
a pressure sensor configured to sense a pressure within the balloon.

2. The delivery system of claim 1 wherein the distal portion of the sheath includes a distal terminus, and wherein the balloon extends distally past the distal terminus in the inflated configuration.

3. The delivery system of claim 1 wherein—
the fluid circuit further includes a connector, a first fluid control device, and a second fluid control device,
the first fluid control device is between the pressure source and the connector and actuatable to fluidly connect the pressure source to the connector,
the pressure sensor is fluidly coupled to the connector, and
the second fluid control device is between the connector and the balloon and actuatable to fluidly connect the connector to the balloon.

4. The delivery system of claim 1 wherein the pressure source is a syringe.

5. The delivery system of claim 1 wherein the elongate sheath is sized and shaped to be advanced into a heart chamber of a patient such that the balloon contacts a wall of the heart chamber.

6. The delivery system of claim 5, further comprising a computing device electrically coupled to the pressure sensor, wherein—
the pressure sensor is configured to convert the sensed pressure within the balloon to an electrical signal,
the computing device is configured to receive the electrical signal from the pressure sensor, and
the computing device is configured to process the electrical signal to determine that the balloon is in contact with the wall of the heart chamber.

7. The delivery system of claim 5, further comprising a computing device electrically coupled to the pressure sensor, wherein—
the pressure sensor is configured to convert the sensed pressure within the balloon to an electrical signal,
the computing device is configured to receive the electrical signal from the pressure sensor, and
the computing device is configured to process the electrical signal to determine a motion of the wall of the heart chamber.

8. The delivery system of claim 5, further comprising a computing device electrically coupled to the pressure sensor, wherein—
the pressure sensor is configured to convert the sensed pressure within the balloon to an electrical signal,
the computing device is configured to receive the electrical signal from the pressure sensor, and
the computing device is configured to process the electrical signal to determine a blood flow characteristic within the heart chamber.

9. The delivery system of claim 1, further comprising an indicator operably coupled to the pressure sensor, wherein the indicator is configured to provide an audible and/or visual indication that the pressure within the balloon is at a selected inflation pressure.

10. A method of sensing contact of a delivery system with a wall of a heart chamber, the method comprising:
advancing a distal portion of a sheath of the delivery system into the heart chamber;
inflating a balloon coupled to the distal portion of the sheath;
advancing the distal portion of the sheath and the balloon toward the wall;
monitoring a pressure within the balloon; and
determining that the delivery system has contacted the wall by sensing a change in pressure within the balloon.

11. The method of claim 10 wherein sensing the change in pressure within the balloon includes sensing an increase in pressure within the balloon.

12. The method of claim 10 wherein monitoring the pressure within the balloon includes fluidly coupling a digital pressure sensor to the balloon.

13. The method of claim 10 wherein inflating the balloon includes fluidly connecting a syringe to the balloon and actuating the syringe to drive a fluid into the balloon, and wherein monitoring the pressure within the balloon further includes fluidly disconnecting the syringe from the balloon after inflating the balloon.

14. The method of claim 10 wherein inflating the balloon includes inflating the balloon to a selected inflation pressure of about 100 pounds per square inch.

15. The method of claim 10 wherein the method further comprises providing a visual and/or auditory indication that the delivery system has contacted the wall.

16. A method of selecting a target site of an endocardial wall for a medical implant, the method comprising:
inflating a balloon coupled to a distal portion of a sheath;
moving the balloon into contact with the endocardial wall at a first site;
monitoring a pressure within the balloon to detect a first delay associated with contraction of the endocardial wall at the first site;
moving the balloon into contact with the endocardial wall at a second site;
monitoring the pressure within the balloon to detect a second delay associated with contraction of the endocardial wall at the second site; and
comparing the first delay and the second delay to select the first site or the second site as the target site.

17. The method of claim 16 wherein comparing the first delay to the second delay to select the first site or the second site as the target site includes determining the greater of the first delay and the second delay and selecting the first site or the second site as the target site based on the greater of the first delay and the second delay.

18. The method of claim 16 wherein monitoring the pressure within the balloon to detect the first delay includes sensing a first decrease in the pressure within the balloon, and wherein monitoring the pressure within the balloon to detect the second delay includes sensing a second decrease in the pressure within the balloon.

19. The method of claim 16 wherein the method further comprises:
sensing a surface electrocardiogram (ECG) signal;
sensing an electromyography (EMG) signal;
when the balloon is in contact with the endocardial wall at the first site, determining a first interval between a first QRS complex of the ECG signal and a first waveform of the EMG signal;
when the balloon is in contact with the endocardial wall at the first site, determining a second interval between a second QRS complex of the ECG signal and a second waveform of the EMG signal; and
comparing the first interval and the second interval to further select the first site or the second site as the target site.

20. The method of claim 16 wherein the endocardial wall is within the left ventricle.
